(19) United States
(12) Patent Application Publication
(10) Pub. No.: US 2005/0164293 A1
(43) Pub. Date: Jul. 28, 2005

(54) Dynamic determination of analytes

(76) Inventors: Peer Stahler, Mannheim (DE); Cord F. Stahler, Weinheim (DE); Andrea Schlauersbach, Aschaffenburg (DE); Manfred Muller, Muenchen (DE); Michael Baum, Heidelberg (DE); Markus Beier, Heidelberg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 11/087,627

(22) Filed: Mar. 24, 2005

Related U.S. Application Data
(63) Continuation of application No. PCT/EP00/11968, filed on Nov. 29, 2000.

(30) Foreign Application Priority Data
Nov. 29, 1999 (DE) 199 57 319.0

(51) Int. Cl. C12Q 1/68

(52) U.S. Cl. 435/6

(57) ABSTRACT

The invention relates to a method for the determination of analytes using carrier chips comprising arrays of different receptors in immobilized form on the surface of said chips. The method is performed dynamically in several cycles. Information obtained in a previous cycle on the modification or alteration of said receptors is used in the following cycle.

The invention relates to a method for determining analytes using support chips which comprise arrays of different receptors in immobilized form on their surface. The method is carried out dynamically in a plurality of cycles, with the information obtained from a preceding cycle being used to modify or change the receptors in the subsequent cycle.

### 1. INTRODUCTION

The collection of biologically relevant information in defined investigation material is of outstanding importance for basic research, medicine, biotechnology and other scientific disciplines. In most cases, genetic information is of central interest. This genetic information consists of an enormous diversity of different nucleic acid sequences, the DNA. This information is utilized in the biological organism, via the production of transcripts of the DNA into RNA, usually for synthesizing proteins. Further valuable information can be obtained from the analysis of RNA and proteins and of the resulting metabolic products.

In order to be able better to understand the principles on which nature acts based on genetics, efficient and reliable decoding of DNA sequences is necessary. Detection of nucleic acids and determination of the sequence of the four bases in the chain of the nucleotides, which is generally referred to as sequencing, provides valuable data for research and applied medicine. In medicine, it has been possible to a greatly increasing extent to develop, and make available to the treating physician, through in vitro diagnosis (IVD) an instrument for determining important parameters of patients. Without this instrument, it would be impossible to diagnose many diseases at a sufficiently early time. Genetic analysis has become established here as an important new method.

It has been possible with close interlinkage of fundamental research and clinical research to trace back and elucidate the molecular causes and (pathological) relationships of some disease states as far as the level of the genetic information. Development of this scientific procedure is, however, still in its infancy, and much more intensive efforts are needed in particular for conversion into therapeutic strategies. Overall, the genomic sciences and the nucleic acid analytical techniques associated therewith have made important contributions both to the understanding of the molecular bases of life and to explaining very complex disease states and pathological processes.

### 2. PRIOR ART

Further essential contributions through molecular analytical methods are to be expected both for the development of therapies and active substances in the field of medicine and for the development of biotechnological approaches. These belong, for example, to the areas of raw materials, environment, methods of manufacture, agriculture and working animal breeding or forensics.

Genetic information is obtained by analysis of nucleic acids, usually in the form of DNA. There are three essential techniques for the analysis of DNA. The principal representative of the first category is the polymerase chain reaction (PCR). This and related methods are used for the selective enzyme-assisted replication (amplification) of nucleic acids by using short flanking strands of known sequence in order to start the enzymatic synthesis of the region in between, usually by means of a polymerase. In this case it is unnecessary for the sequence of this region to be known in detail. The mechanism thus permits, on the basis of a small segment of information (the flanking DNA strands), the selective replication of a particular DNA section so that this replicated DNA strand is available in large quantities for further studies and analyses.

Electrophoresis is the second basic technique in use. This comprises a technique for separating DNA molecules on the basis of their size. The separation takes place in an electric field which forces the DNA molecules to migrate. Movement in the electric field is impeded as a function of the molecular size by suitable media such as, for example, crosslinked gels, so that small molecules, and thus shorter DNA fragments, migrate more quickly than do longer ones. Electrophoresis is the most important established method for DNA sequencing and moreover for many methods for purifying and analyzing DNA. The most widely used method is slab gel electrophoresis, although this is increasingly being displaced by capillary gel electrophoresis in the area of high throughput sequencing.

The third method comprises analysis of nucleic acids by so-called hybridization. This entails use of a DNA probe of known sequence in order to identify a complementary nucleic acid, usually in the presence of a complex mixture of very many DNA and RNA molecules. The matching strands bind together stably and very specifically.

The three basic techniques are frequently combined, in that, for example, the sample material for a hybridization experiment is selectively replicated by PCR beforehand.

Sequence analysis on a DNA support chip likewise utilizes the principle of hybridization of mutually matching DNA strands. The development of DNA support chips or DNA arrays signifies an extreme parallelization and miniaturization of the format of hybridization experiments. DNA in a sample can bind only to those of the sites on the DNA immobilized on the support where there is sequence agreement of the two DNA strands. It is possible with the aid of the immobilized DNA on the chip selectively to detect the complementary DNA in the sample. In this way, for example, mutations in the sample material are recognized from the pattern produced on the support after the hybridization.

A considerable restriction in the processing of very complex genetic information using such a support is the access to this information due to the limited number of measurement points on the support. One such measurement point is a reaction zone in which DNA molecules are synthesized as specific reactants, called probes, in the production of the support.

There are in principle two possibilities for larger data throughput: the first consists of increasing the number of measurement points on a reaction support. However, the number of possible probes still remains small compared with the biological diversity and minimal in relation to the statistical diversity. The second is based on increasing the number of different probes which the system is able to generate per unit time (and for the money employed) and provide for hybridization. The second possibility has something to do with the number of variants generated in the system and made available for the analysis (data throughput).

With the concept of genetic information it is necessary to distinguish between unknown sequences which are to be decoded for the first time (this is generally referred to as sequencing, also de novo sequencing) and known sequences which are to be identified for reasons other than initial decoding. Examples of such other reasons are the investigation of the expression of genes or the verification of the sequence of a DNA section of interest in an individual. This may take place, for example, in order to compare the individual sequence with a standard, as in the mutation analysis of cancer cells and the typing of HIV viruses.

Electrophoretic methods almost exclusively have been used to date for de novo sequencing. The fastest is capillary electrophoresis.

Supports have scarcely played a part in de novo sequencing to date. This is because of limitations in principle: to obtain information by comparison of sequences it is necessary to provide probes on the support. A large number of different probes (variants) is needed for processing unknown material. No method known to date is able to generate the necessary numbers of variants for efficient sequencing by comparison of sequences of very large amounts of DNA. Such very large amounts of DNA are present, for example, in the determination of the sequences of whole genomes.

Essentially two methods have been known to date for producing supports. In the first production method, the finished probes are produced singly either in a synthesizer (chemically) or from isolated DNA (enzymatically), and they are then applied in the form of tiny drops to the surface of the support, specifically each individual type of probes on a single measurement point. The most widely used method for this is derived from the technique of ink jet printing, and thus these methods are embraced by the generic term of spotting. Also widely used are methods using needles.

Only through the micropositioning of printing head or needle is it possible subsequently for a signal on the chip to be assigned to a particular probe (array with lines and columns). The spotting equipment must operate in an appropriately accurate manner.

In the second method, the DNA probes are produced directly on the chip, in particular by site-specific chemistry (in situ synthesis). There are at present two methods for this.

The first operates with the spotting equipment described above, but with the difference that the tiny drops contain appropriate synthetic chemicals so that the spatially resolved chemistry can be operated by the micropositioning of these chemicals. The technology permits any desired programming of the sequence of the resulting probes. However, the throughput, which is the number of probes per unit time, is as yet not really high enough for conversion of large amounts of genetic information, and the size of the measurement points is limited.

It is possible to produce very many more measuring points per unit time with the second method: parallel synthesis of the probes using light-dependent chemistry. This has been used already to synthesize more than 100 000 measuring points per chip in a few hours.

The method is operated with two technical solutions for the illumination. The first uses photolithographic masks and generates through the highly developed optical system a very large number of measurement points on the DNA support. However, the choice of the probe sequence is very limited because corresponding masks have to be produced. This method of production is therefore not very suitable for the method of the invention. Considerably more promising are methods with a freely programmable probe sequence, which operate on the basis of appropriately controllable light sources. Methods of this type for producing probes on a support are described inter alia in the patent applications DE 198 39 254.0, DE 198 39 256.7, DE 199 07 080.6, DE 199 24 327.1, DE 199 40 749.5, PCT/EP99/06316 and PCT/EP99/06317.

In summary, it can be said that with the techniques which have been established to date for processing larger amounts of genetic information of entirely or partly unknown composition, namely electrophoresis methods and biochip supports, there is a limitation on the throughput. High throughput projects for new sequencing have to date relied on grading according to size using electrophoresis (inter alia the Human Genome Project HUGO). Improvements through miniaturization and parallelization, but no breakthroughs, are to be expected with this, because the technique as such cannot be modified. Electrophoresis is capable of most of the applications of biochips such as, for example, expression patterns or mutation screening only very much more slowly or not at all. Biochips disclosed to date are in turn unsuitable for new sequencing, the emphasis being on the highly parallel processing of material based on known sequences (inter alia in the form of synthetic oligonucleotides as probes). These biochips are not capable in an efficient and economic manner of a dynamic or evolutional selection, an information cycle or a selection process. Both formats have a limited throughput of genetic information. In order to increase this throughput it is necessary to develop new approaches. The method of the invention is such an approach, which can be employed for nucleic acids, but also for other classes of substances such as peptides, proteins and other organic molecules.

### 3. SUBJECT MATTER OF THE INVENTION

The invention relates to a method for determining analytes in a sample comprising the steps:
(a) carrying out a first determination cycle comprising:
   (i) providing a support with a surface which comprises immobilized receptors on a plurality of predetermined zones, where the receptors in individual zones each have a different analyte specificity,
   (ii) contacting the sample containing the analytes to be determined with the support under conditions with which binding is possible between the analytes to be determined and receptors specific therefore on the support, and
   (iii) identifying those predetermined zones on the support onto which binding has taken place in step (ii),
(b) carrying out a subsequent determination cycle comprising:
   (i) providing another support with a surface which comprises immobilized receptors on a plurality of predetermined zones, where the receptors in individual zones each have a different analyte specificity, where the receptors selected for the other support have been observed in a preceding cycle to be associated with a predetermined characteristic signal, and where the selected receptors or/and the conditions of the receptor-analyte binding are changed by comparison with a preceding determination cycle,
   (ii) repeating step (a) (ii) with the other support and
   (iii) repeating step (a) (iii) with the other support and
(c) where appropriate carrying out one or more further subsequent determination cycles in each case selecting and changing the receptors as in step (b) (i) until sufficient information is available about the analytes to be determined or/and the selected receptors provide a signal meeting predetermined criteria.

Support or reaction support is intended to mean in this connection both open and closed supports. Open supports may be planar (e.g. laboratory cover slide), but may also have a special shape (e.g. dish-shaped). With all open supports, the surface is to be understood to be an area on the outside of the support. Closed supports have an interior structure which comprises, for example, microchannels, reaction chambers or/and capillaries. In this case, the surfaces of the support are to be understood to be the surfaces of two- or three-dimensional microstructures in the interior of the support. Combination of interior closed and exterior open surfaces in one support is of course also conceivable. Examples of materials used for supports are glass such as Pyrex, Ubk7, B270, Foturan, silicon and silicon derivatives, plastics such as PVC, COC or Teflon, and Kalrez.

A flexible, rapid and fully automatic method for array generation with integrated detection in a logical system as described in, for example, DE 199 24 327.1, DE 199 40 749.5 and PCT/EP99/06317 makes it possible to obtain within a short time, through analysis of the data of one array, the information necessary to construct a new array (information cycle). This information cycle allows automatic adaptation of the next analysis through a selection of suitable polymer probes for the new assay. It is moreover possible by taking account of the result obtained to restrict the scope of the objective in favor of greater specificity or modulate the direction of the objective. A further possibility through altering receptors is also to follow partly specific analyte bindings, e.g. bindings of analyte groups which are “similar” to one another, until an accurate assignment of the analyte in the sample is possible. Thus, compared with the methods in use to date, some of which have been described above, a multiple of the amount of information to date is turned over with relatively little effort and moreover valuable information is collected.

In the method of the invention this new format is utilized for DNA arrays and further developed by producing the specific probes on or in the support flexibly by means of in situ synthesis so that a flow of information is possible. Every new synthesis of the array can take account of the results of a preceding experiment. A suitable choice of probes in relation to their length, sequence and distribution on the reaction support and a feedback of the system with integrated signal evaluation makes efficient processing of genetic information possible.

The spatial and temporal coupling of production and evaluation (analysis) of the arrays, preferably in one instrument, allows the process and the use of information cycles to be automated easily. In this case, the user fixes the criteria for the selection (selection criteria).

The method of the invention is suitable in principle for determining any analytes such as those which may be present in sample material, in particular samples of biological origin. A determination of nucleic acid analytes is particularly preferred. However, it is also possible to determine proteins, peptides, glycoproteins, medicaments, drugs, metabolic intermediates etc.

In a preferred embodiment, the receptors used are polymer probes, in particular nucleic acids or analogs thereof, e.g. peptide nucleic acids (PNA) or locked nucleic acids (LNA). However, the use of other types of receptors is also conceivable, or a combination of several types of receptors, e.g. peptides, proteins, saccharides, lipids or other organic or inorganic compounds which can be disposed appropriately in an array.

The binding of the analytes to receptors on the respective zones on the receptor surface is preferably detected via labeling groups. The labeling groups may in this case be bound directly or indirectly, e.g. via soluble analyte-specific receptors, to the analyte. The labeling groups preferably used are optically detectable, e.g. by fluorescence, refraction, luminescence or absorption. Preferred examples of labeling groups are fluorescent groups or optically detectable metal particles, e.g. gold particles.

The immediate evaluation and subsequent utilization of the collected data makes the method described below a learning process with the aid of which it is possible inter alia to determine for example in a short time all 25-nucleotide long nucleic acids (25-mers) in a predetermined sequence without the need to synthesize them in their diversity (4²⁵=1.125899907×10¹⁵).

This can be utilized in the preferred embodiment in order to identify in an unknown sequence or a mixture of unknown sequences a number of part-sequences with little or no redundancy, so that finally the amount of the actually present sequences is separated in a type of filter from the amount of part-sequences possible theoretically in a nucleic acid.

Rapid and economical and automatable selection of a selected set of polymer probes which corresponds to a subpopulation of genes from a complete genome which is where appropriate already deposited as sequence information in a database is also possible for expression analysis.

Another important use is the empirically assisted selection of sets of polymer probes with defined properties. These properties may in the case of nucleic acid probes be, for example, binding characteristics, melting point, accessibility to target molecules (targets) or other properties which can be used for specific selection.

In another embodiment there is variation not of the composition of the receptors but of the geometry of the arrays during the method. This may be, for example, the size of the measurement field on which the polymer probes are synthesized (synthesis sites). Optimization is also possible in this case according to particular criteria based on the corresponding signal.

### 4. DETAILED DESCRIPTION OF THE INVENTION

### 4.1 Numerical Ratios

In any sequence consisting of m nucleotides it is possible for a maximum of m-n+1 part-sequences of length n to occur. This means that for any complete sequence length m there is a specific sequence length n for which the number of all possible n-mers (4ⁿ) exceeds the number m-n+1 of the possible part-sequences of length n in the complete sequence.

In the E. coli genome for example, which consists of about 4.6×10⁶ nucleotides, it is thus possible for a maximum of about 4.6×10⁶ sequence sections of any length n to occur. If n=12 is chosen, the number of all 12-mers is 4¹²=16777216, which is distinctly larger than the maximum number of 12-mers occurring in the E. coli genome. It is thus completely impossible all 12-mers and therefore also for all longer (n+1)-, (n+2)-mers etc. to occur in this genome.

TABLE 1
Probabilities (in %) of the occurrence of an
n-mer in a sequence of length m

| n/m | 500 | 1000 | 10000 | 50000 | 100000 | 500000 | 1000000 | 5000000 | 10000000 |
|---|---|---|---|---|---|---|---|---|---|
| 1 | 100.0000 | 100.0000 | 100.0000 | 100.0000 | 100.0000 | 100.0000 | 100.0000 | 100.0000 | 100.0000 |
| 2 | 31.1875 | 62.4375 | 100.0000 | 100.0000 | 100.0000 | 100.0000 | 100.0000 | 100.0000 | 100.0000 |
| 3 | 7.7813 | 15.5938 | 100.0000 | 100.0000 | 100.0000 | 100.0000 | 100.0000 | 100.0000 | 100.0000 |
| 4 | 1.9414 | 3.8945 | 39.0508 | 100.0000 | 100.0000 | 100.0000 | 100.0000 | 100.0000 | 100.0000 |
| 5 | 0.4844 | 0.9727 | 9.7617 | 48.8242 | 97.6523 | 100.0000 | 100.0000 | 100.0000 | 100.0000 |
| 6 | 0.1208 | 0.2429 | 2.4402 | 12.2058 | 24.4128 | 100.0000 | 100.0000 | 100.0000 | 100.0000 |
| 7 | 0.0302 | 0.0607 | 0.6100 | 3.0514 | 6.1031 | 30.5172 | 61.0348 | 100.0000 | 100.0000 |
| 8 | 0.0075 | 0.0152 | 0.1525 | 0.7628 | 1.5258 | 7.6293 | 15.2587 | 76.2938 | 100.0000 |
| 9 | 0.0019 | 0.0038 | 0.0381 | 0.1907 | 0.3814 | 1.9073 | 3.8147 | 19.0735 | 38.1469 |
| 10 | 0.0005 | 0.0009 | 0.0095 | 0.0477 | 0.0954 | 0.4768 | 0.9537 | 4.7684 | 9.5367 |
| 11 | 0.0001 | 0.0002 | 0.0024 | 0.0119 | 0.0238 | 0.1192 | 0.2384 | 1.1921 | 2.3842 |
| 12 | 0.0000 | 0.0001 | 0.0000 | 0.0030 | 0.0060 | 0.0298 | 0.0596 | 0.2980 | 0.5960 |
| 13 | 0.0000 | 0.0000 | 0.0000 | 0.0007 | 0.0015 | 0.0075 | 0.0149 | 0.0745 | 0.1490 |
| 14 | 0.0000 | 0.0000 | 0.0000 | 0.0002 | 0.0004 | 0.0019 | 0.0037 | 0.0186 | 0.0373 |
| 15 | 0.0000 | 0.0000 | 0.0000 | 0.0000 | 0.0001 | 0.0005 | 0.0009 | 0.0047 | 0.0093 |
| 16 | 0.0000 | 0.0000 | 0.0000 | 0.0000 | 0.0000 | 0.0001 | 0.0002 | 0.0012 | 0.0023 |
| 17 | 0.0000 | 0.0000 | 0.0000 | 0.0000 | 0.0000 | 0.0000 | 0.0001 | 0.0003 | 0.0006 |
| 18 | 0.0000 | 0.0000 | 0.0000 | 0.0000 | 0.0000 | 0.0000 | 0.0000 | 0.0001 | 0.0001 |
| 19 | 0.0000 | 0.0000 | 0.0000 | 0.0000 | 0.0000 | 0.0000 | 0.0000 | 0.0000 | 0.0000 |

The facts described above are depicted clearly in table 1. For any specifically chosen n-mer in each case the probability with which it occurs in a sequence of length m is calculated, assuming for simplicity an equal distribution of all n-mers. The probability in this case is determined by the length m of the sequence, the length n of the observed part-sequence and the number of all possible sequences of length n.

It is clearly evident that the values for the probability become very small as soon as the length n of the observed part-sequence becomes so large that it is no longer possible for all n-mers to occur in the sequence of length m. This relationship between the sequence section length n, the sequence length m and the maximum number of part-sequences of length n contained in the sequence of length m is depicted in table 2. In any sequence which is shorter than the value indicated for m it is possible in each case for only some of all the possible sections of the indicated length n to occur.

TABLE 2
Relationship between the sequence length m and
the maximum possible number of different n-mers
contained therein

| | Sequence length | n-mers in the sequence |
|---|---|---|
| n | m | m − 4\hat{ }n + 1 |
| 3 | 66 | 64 |
| 5 | 1028 | 1024 |
| 6 | 4101 | 4096 |
| 7 | 16390 | 16384 |
| 8 | 65543 | 65536 |
| 9 | 262152 | 262144 |
| 10 | 1048585 | 1048576 |
| 12 | 16777227 | 16777216 |
| 13 | 67108876 | 67108864 |
| 14 | 268435469 | 268435456 |
| 15 | 1073741838 | 1073741824 |
| 16 | 4294967311 | 4294967296 |
| 17 | 17179869200 | 17179869184 |
| 18 | 68719476753 | 68719476736 |
| 19 | 2.74878E+11 | 2.74878E+11 |
| 20 | 1.09951E+12 | 1.09951E+12 |
| 25 | 1.1259E+15 | 1.1259E+15 |

For an array on which all probes of length s are synthesized, the above considerations mean, for example, that after hybridization with the initial sequence under suitably chosen synthesis and hybridization conditions it is never possible for all the probes to provide a signal. With a suitable choice of the probe length s it is possible to predict an upper limit for the number of signal-emitting probes, and this is determined by s≧m+1-SP where SP is the number of signal-emitting probes. Such an upper limit may be important for example in sequencing to determine the starting probe length.

### 4.2.1 Methods

As described above, only a fraction of the possible nucleotide combinations of a length n can be utilized in each sequence, and thus it is sensible also to synthesize only a selection of these combinations on the arrays in order to investigate the required sequence.

The length s of the starting probes, i.e. of the probes on the first array, can be chosen according to various criteria which emerge from use. For the method mentioned above, this may be, for example, the maximum desired number of signal-emitting probes. If all possible combinations of a certain length s are to be synthesized on the first array, then, for example, the size of the available array is a criterion for determining the probe length, because the required number of sites (4^{s}) must not exceed the number of sites available.

For other applications it is conceivable inter alia that only probes with identical properties, e.g. with the same start or end sequence, are of interest, and this in turn reduces the number of possible probes.

All probes of the chosen length and properties are then synthesized on the array employed in the first determination cycle, and the sequence to be investigated is hybridized with them. As described above, it is unlikely that signals will be emitted from all sites after the hybridization because, with a suitable choice of the probe length, not all probe sequences can occur in the initial sequence. In addition, some probe sequences occur more frequently in the initial sequence, which leads to multiple binding to individual sites and thus reduces the number of signals.

All the sites relevant for the particular use are varied on a new array. This can take place in various ways.

### 4.2.2 Variation Through Extension of the Probes/Iterative Probe Construction

One possibility for varying the probes on a new array is to change their length, i.e. make them more specific by extension by one or more nucleotides. For this purpose, all probes which have generated a signal on the previous array are synthesized on a new array and each extended by all the nucleotide building blocks relevant for the investigated type of sequence. This means for an investigation of DNA/RNA sequences for example an extension of each probe by the four nucleotides adenine, thymine, guanine and cytosine. In this case, for each site on the previous array, four sites are required on the new array, one for each of the four nucleotides, see table 3. In all other cases the number of sites on the new array per site on the previous array is the number of building blocks by which the probes can be extended.

TABLE 3
Example of probe extension for DNA/RNA
sequences

New sites

| Old site | A | C | G | T |
|---|---|---|---|---|
| N | N | N | N | N |
| N | N | N | N | N |
| N | N | N | N | N |
| N | N | N | N | N |
| N | N | N | N | N |
| N | N | N | N | N |
| N | N | N | N | N |
| N | N | N | N | N |
| N | N | N | N | N |
| N | N | N | N | N |

The initial sequence is hybridized with the newly synthesized probes in the subsequent array; not all probes will emit a signal after this step either. The relevant probes are constructed on a new array and extended further, and thus the new number of sites is always four times the number of signals on the previous array. This procedure is continued until a previously fixed maximum probe length is reached.

### 4.2.3 Filter Effect of Iterative Construction

The iterative construction described herein of the probes relevant for investigating the initial sequence acts like a filter which, irrespective of the probe length, rejects the probes which have provided no signal. On each new array the number of probes then made available equals the possibilities for extending a successful probe. After a specific probe length which depends on the length and nature of the initial sequence has been exceeded, the number of signals on the following arrays will not increase further, and thus the number of sites remains approximately constant. The method thus makes it possible for very specific probes which are important for the particular use to be selected and for only these to be synthesized. Each sample sequence can thus be compared with the diversity of oligonucleotides of specific lengths without needing to generate all possible combinations of this length, and thus there is no restriction in the diversity of combinations on investigation of the initial sequences.

The criteria for a successful probe can moreover be varied as parameters and be fixed depending on the aims of the optimization. Such a fixing might also be the selection of a proportion of the probes which show a particular signal, that is to say, for example, exceed a certain fixed threshold. This threshold may in turn be made dependent on the overall signal so that, for example, the 25% of polymer probes with the highest signal are categorized as successful. Other criteria would be, for example, the kinetics of the binding reaction or the specificity of the binding.

A sequence of 50 000 nucleotides contains, as described in 4.1, a maximum of 50 000 different part-sequences of a length n. If in this case n=8 is chosen, there are more 8-mers (4⁸=65536) than can occur in this sequence. If all 8-mers are synthesized on the first array, signals cannot be emitted by all the probes after the hybridization. The relevant probes are then extended on the following array, and the required number of sites on the following array is thus 4× number of signals but, in any event, less than 4×4⁸=262144. In no case will the number of sites required on the following arrays exceed this value.

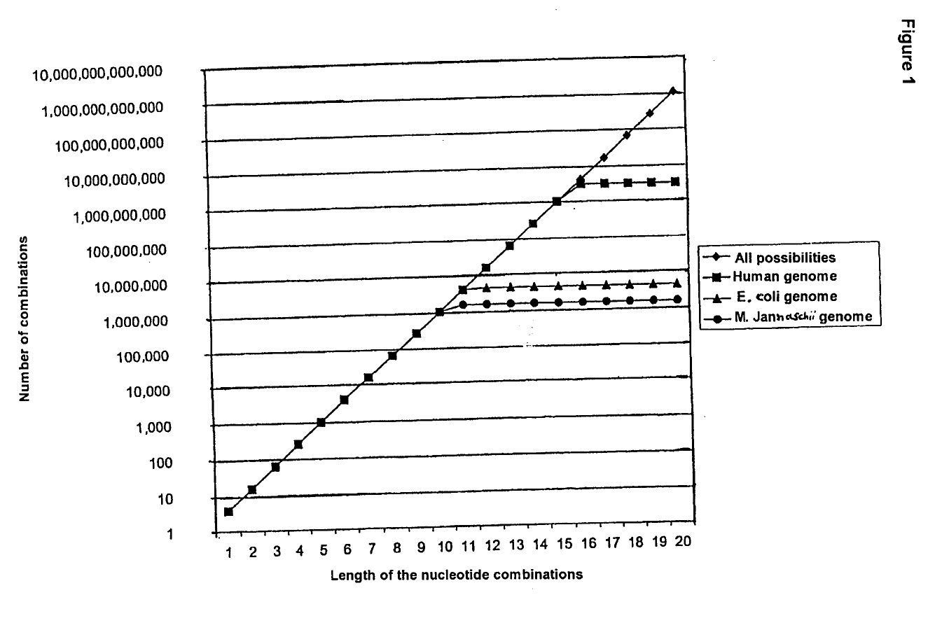

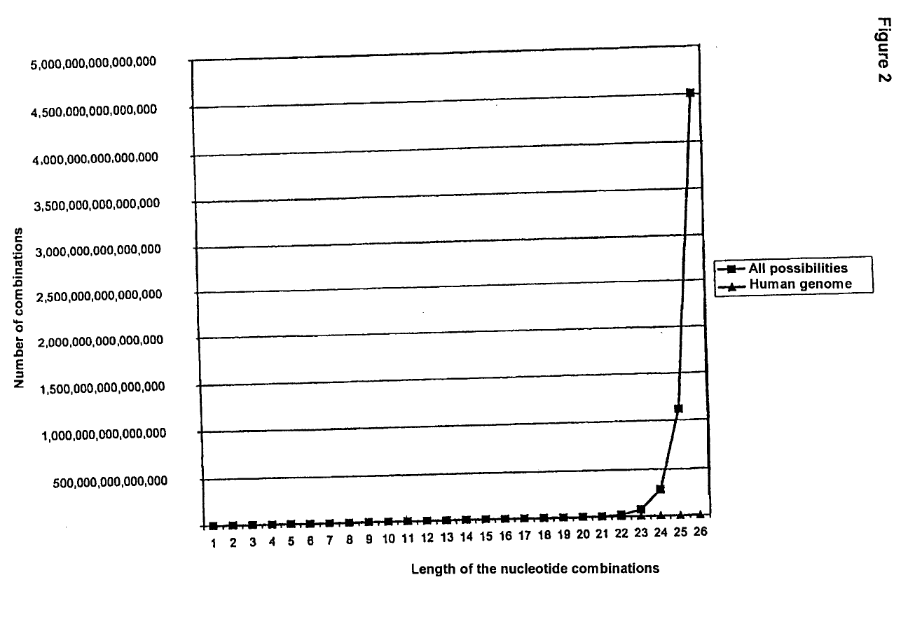

FIGS. 1 and 2 show the relationship between the number of all the possible sequences of length n and the number of potentially possible different part-sequences which may occur in the human genome, in the genome of E. coli and in the M. jannaschii genome. The number of all possible combinations (4ⁿ) increases exponentially, while the possible number of part-sequences in the genomes does not increase further when a specific length is reached. Nature makes use of only a few of the available possibilities, and these can be detected with a learning system on which the method described herein is based.

If signals in some iteration steps cannot be evaluated unambiguously, these probes can be regarded as relevant probes and be constructed further on the new arrays. As the length of the probes increases, the hybridization becomes more specific and the information, as expected, becomes clearer.

### 4.2.4 Optimization and Verification of the Results by Varying the Probes

In addition to the extension described above, probes can also be varied in other ways from one array to the next. Thus—in the case of polymeric probes—variation within the probe sequence is also possible through substitution of individual building blocks, e.g. nucleotides, by other building blocks. A further possibility is to vary the position or/and the density of receptors on the support area. A variation in the nature of the coupling of receptors to the support surface, e.g. in relation to the linker molecules used for this, is also possible. In addition, the conditions for binding between analyte and receptor can be varied in consecutive determination cycles, it being possible, for example with nucleic acid analytes, to vary the hybridization conditions (e.g. salt content, temperature, fluid movement or other parameters). Finally, the synthesis conditions during construction of the receptor, e.g. in the coupling of complete receptors and, in particular, in the construction of the receptors from a plurality of synthon building blocks, can also be varied.

Thus, for example, the position of the site or the density of the sites may have an effect on the hybridization or/and synthesis conditions, so that unambiguous assignment of the result obtained after the hybridization is not possible. It may be possible by choosing a new site position or an altered site density on the following array to generate a better positive signal or confirm the absence of a signal. This makes it possible inter alia to collect during the method experience about the hybridization and synthesis conditions of the individual probes. The results can be deposited for example in a database in order to be reused with a similar problem points. The generated data can be used to optimize the system for every problem arising so that, for example, it is possible over the course of time, or in tests designed for this purpose, for there to be selection of probes with which the same problem arising for different sample material can be solved.

If only selected probes are synthesized on an array, it is possible for probes which appear relevant to be altered only at a few places in the sequence in the next step, that is to say for a few nucleotides to be replaced with others. The probes suitable for such a modification must be established separately for each application.

### 4.3 Exemplary Embodiments

Two examples are intended to illustrate how the method described above can be used, for example, for determining all n-mers of specific lengths in a sequence without the need to compare the sequence to be investigated with all existing n-mers.

In the first example, the M. jannaschii genome which consists of about 1.6 million nucleotides is investigated. A simulation is used initially to determine all 9-mers of this genome (single-stranded for simplification). Of 262 144 possible combinations of a length of 9 nucleotides, 177 167 combinations occur in one strand of the investigated genome. In the next step, all the relevant probes are extended; after renewed hybridization, signals are emitted by 436 325 of the 708 668 sites on the new array. In the simulation, this procedure is repeated up to a length of 13 nucleotides. After the hybridization in the last step, signals are emitted by 1 441 322 sites. This is only a fraction of the possible total of 67 108 864 combinations of a length of 13 nucleotides.

In total, up to about 1.6 million different part-sequences of a length n may occur in one strand of the M. jannaschii genome. The method approaches this upper limit with every step but can never exceed it. This means inter alia that more than 6.4 million sites will not be needed in any step, which is a relatively small number compared with the diversity of all the possible combinations.

In the second example, a human gene 188 642 nucleotides long is investigated. For simplification, a single strand is chosen in this simulation too.

In the first step, all possible probes with a length of 6 nucleotides (4096) are synthesized on an array. The probability with which a probe sequence occurs more than once in the sequence to be investigated is 100%, which is why signals are emitted from all sites after the hybridization, and thus the chosen length of the probe was too short. In the next step it is therefore necessary to synthesize all, that is to say 16 384, 7-mers. After the hybridization there are 14 803 relevant probes, which are synthesized and extended on a new array. This procedure is repeated up to a probe length of 20 nucleotides. After the last hybridization, signals are emitted from 180 362 sites. During the method, the number of relevant probes approaches the maximum possible number of approximately 188 600 but, as in the first example, this number cannot be exceeded.

Thus, the method of the invention makes it possible to determine part-sequences of a specific length without the need to generate all sequences of this length.

### 4.4 Sample Preparation

The method of the invention can be carried out both with single-stranded RNA or DNA (ssRNA or ssDNA) and with double-stranded nucleic acids, e.g. dsRNA and dsDNA. The nucleic acids are for this purpose isolated according to the state of the art from viruses, bacteria, plants, animals or humans, or may be derived from other sources.

Single-stranded nucleic acids are generated in the majority of cases by specific in vitro methods starting from dsDNA. These include, for example, asymmetric PCR (generates ssDNA), PCR with derivatized primers which make selective hydrolysis of a single strand in the PCR product possible, or transcription by RNA polymerases (generates ssRNA). The templates which can be employed in the transcription are, besides uncloned single-stranded DNA, in particular also dsDNA cloned into specific vectors, (e.g. plasmid vectors with a promoter; plasmid vectors with two differently oriented promoters for one particular or two different RNA polymerases). The insert DNA cloned into the plasmids, or the DNA template employed in the PCR, can be isolated on the one hand from viruses, bacteria, plants, animals or humans, on the other hand, however, in principle also be generated in vitro by reverse transcription, RNaseH treatment and subsequent amplification (e.g. by PCR) from ssRNA. Suitable RNA templates in this case are, besides rRNAs, tRNAs, mRNAs and snRNAs, also transcripts generated in vitro (produced, for example, by transcription with SP6, T3 or T7 RNA polymerase). Other methods are also conceivable for the skilled worker.

Double-stranded nucleic acids can be obtained, for example, from dsDNA. This dsDNA can on the one hand be isolated as genomic, chromosomal DNA, as extrachromosomal element (e.g. as plasmid) or as constituent of cell organelles from viruses, bacteria, animals, plants or humans, but on the other hand in principle also be generated in vitro by reverse transcription, RNaseH treatment and subsequent amplification (e.g. by PCR) from ssRNA. RNA templates which can be employed in this case are, besides rRNAs, tRNAs, mRNAs and snRNAs, once again transcripts generated in vitro (produced, for example, by transcription with SP3, T3 or T7 RNA polymerase).

The nucleic acids intended for the method are preferably fragmented in a sequence-specific or/and non-sequence-specific manner (e.g. by (non)-sequence-specific enzymes, ultrasound or shear forces), the aim being a predetermined, e.g. essentially homogeneous, distribution of the lengths of the fragments/hydrolysis products. If the predetermined distribution of the lengths of the fragments is not achieved initially, it is possible subsequently to carry out a fractionation by length, e.g. by gel electrophoretic or/and chromatographic methods, in order to obtain the desired distribution of lengths. There may, however, also be applications in which a defined fragmentation is carried out, e.g. using sequence-specific enzymes or ribozymes.

The resulting fragments are appropriately labeled, e.g. with fluorescent agents; other possibilities are the incorporation of radioactive isotopes, light-refracting particles or enzymatic labels such as peroxidase. The labeling moreover preferably takes place at the ends of the fragments (terminal labeling). 3′-Terminal labelings can be carried out by using suitable synthons, e.g. with terminal transferase or T4 RNA ligase. If RNA transcripts generated in vitro are employed for the fragmentation, the labeling can also take place before the fragmentation through labeled nucleotides employed in the transcription (internal labeling). Further methods such as nick translation are known to the skilled worker.

The labeled, fragmented nucleic acids can then be hybridized in a suitable hybridization solution with the oligonucleotide array.

### 5. APPLICATIONS

The method of the invention can be utilized in one embodiment for the analysis of differential expression. For this purpose, two samples A and B are obtained from different cells which are to be compared with one another. In this connection, A might be a normal cell and B a cancer cell. Any other differences are possible.

The samples are then characterized with the aid of dynamic learning arrays, and the probes categorized as negative, i.e. have emitted no signal by definition, are those with sufficiently similar or identical representation in the two samples. The probes which are followed up are given away those with which a signal was to be seen with only one of the two samples. Thus, there is selection of increasingly specific probes which find complementary sequences only in one of the two samples. With a length of 25-30 nucleotides, such a probe is highly specific for humans, even taking account of all the genes present (which are never expressed all at the same time) which comprise only 1-10% of the genomic DNA. The selected probes thus become markers for differentially expressed genes or else at least splice variants. At the same time, however, it is also possible to see a correlate for ESTs therein, because with an appropriate length of probe it would be possible to determine 30-40 base pairs of the sequence. If 30mers are determined as differential markers for the human genome, then it is very likely that this length of the probe is sufficient Go allow unambiguous identification of the corresponding mRNA molecule.

The probes can be utilized in a further step as capture probes for the specific isolation of the corresponding mRNA population. It is possible in this way to obtain material which is available for further investigations such as sequencing or cloning.

It is thus possible on the one hand to fish clones out of a library, e.g. using established methods such as blots and filters from libraries in bacteria, yeasts or other suitable cells. On the other hand, these oligo-ESTs can also be utilized for deciphering further parts of the sequence from this point using known methods such as primer walking or other methods.

In one variant, the method of the invention can be utilized for optimizing suitable capture probes in an appropriate learning method. This can take place, for example, with a view to their specificity or/and their accessibility for the target molecules.

Other oligonucleotides can also be optimized in the method of the invention for properties such as a particular function, the specificity of binding or/and accessibility to the target molecule. Examples of such oligonucleotides are antisense molecules and ribozymes.

In another variant of the method, phage libraries or similar biologically functional libraries are selected by means of the method of the invention with particular optimization aims. The advantage of such a use is the parallel optimization of the probes on the solid phase and the selection of a population from the library. It is thus possible to expedite optimization processes.

In yet a further variant it is possible to use the differential probes without further characterization on further arrays in order thus to investigate further samples, e.g. cells assigned to a similar disease state. It is thus possible without further work such as cloning, functional studies etc. to produce an association or establish a combination of probes which appears appropriate for diagnostic purposes. This makes a large part of a screening approach with high throughput possible with relatively small expenditure on molecular biological and biochemical experiments, and only interesting probes or oligo-ESTs are included in further studies.

One aspect of the described applications is that substantially undefined material can be additionally screened efficiently, without previous knowledge about the sequence of the nucleic acid present therein, for differentially expressed or differentially represented probes and thus, where appropriate, genes or splice products. Only one comparative sample, against which the differentiation is carried out, is required.

Another substantial advantage of the described procedure is that the selection process itself includes the optimization of the probes for stable hybridization, accessibility of the target sequence and distinctness of the signal. It is virtually inherent to the system that the selected probes are most suitable for a distinct signal and are moreover highly specific.

In a further embodiment, the described mechanisms are employed to compare genomic DNA in two samples. It is thus possible to identify, for example, chromosomal aberrations such as deletions etc.

In another embodiment, genomic DNA populations are compared in order to identify so-called single nucleotide polymorphisms (SNP). It may for this purpose be expedient to compare the DNA from two or more sample sources. It may also be of interest for the comparison process in the case of known SNPs to examine the content of two or more genomes for these SNPs in order to find the different SNPs in an automated method.

A further aspect of the invention is the possibility of optimizing the physicochemical properties of the polymer probes. These include, for example, the length of the linker molecule connecting a receptor to the solid phase, its charge or other characteristics of the linker which influence the receptor binding event. It is also possible for effects due to interaction of receptors on adjacent fields and the different accessibility of sample material for the receptors to be systematically optimized.

A further physicochemical property which could be optimized is the melting temperature or duplex stability under certain conditions such as, for example, the salt content in the buffer.

This process is then suitable in principle for developing libraries of polymer probes with particular properties. One example would be a library of oligo probes which are 25-30 bases long and have their melting point (defined as Tm) at a predetermined temperature, e.g. 35° C. An empirically developed library of this type is of very great value for selecting appropriate oligo probes for different applications, in particular for application as probes on an array. The library can be used when developing a new array for a particular objective, e.g. detection of the expression of a small selection of genes from a relatively large genome such as the human genome, in order rapidly to include suitable and empirically validated probes in the selection process.

Other libraries may be selected according to a particular length. It is possible in turn to mix probes from different libraries. The selection process may in this case take place so that the maximum possible variance of oligomers is built up starting from a particular number of sites. These would be in the case of 64 000 sites in roughly all 8mers. This array is hybridized with a mixture of all 8mers as sample, and the 25% of probes with the strongest signal are selected. These successful probes are then extended to 9mers in a new array. It is thus possible to produce a library of oligomers of length n, after n-initial length information cycles, which consist of b=(number of sites) possible members. This solves many of the problems which are known to the skilled worker and are associated with the purely theoretical prediction of suitable oligo probes by an empirical method. For a large number b it is also possible to construct a generation of oligo probes in parallel or successively in different reaction supports. It would thus be possible to start with n=10 with about 1 million sites.

The method is moreover suitable for optimizing the production process or for comparative investigations of the quality of synthesis.

Another aspect of the invention is the design of diagnostic systems, e.g. of individualized or/and multistage diagnostic systems which produce an analytical answer likewise in learning cycles and examine the sample material for example in two or more cycles. In this case, the first round or the first array might serve for a type of “pre-scan” in analogy to an image scanner, with this being followed by a deeper search at the points recognized as relevant. Thus, in a specific application, it would be possible first to identify the expression status in order then to identify in detail the sequence of those genes which show a deviation, or to determine particular known aberrations, mutations or SNPs. It is possible in this case to compare the samples for example with a standard, and the nature of the further analysis can then follow where appropriate from this comparison, e.g. selection of diagnostic combinations of polymer probes on the next array. In a further application it is conceivable then to develop from this approach “dynamic” tests with the aim of sending the sample material through a plurality of loops of modification or optimization of the array until, for example, the diagnostic answer exceeds a statistical threshold (significance etc.).

Overall, a flexible system which operates in an evolutional manner based on selection processes, like the method of the invention, is more suitable than rigid arrays for confronting the plasticity of life and its manifestations with a plasticity of the analytical tools and objectives in order thus also to gain worthwhile information with limited effort in the light of the mass of information in biological systems.

1. A method for determining analytes in a sample comprising the steps:
(a) carrying out a first determination cycle comprising:
(i) providing a closed support with a surface on an interior structure which comprises immobilized receptors on a plurality of predetermined zones, where the receptors in individual predetermined zones each have a different analyte specificity,
(ii) contacting the sample containing the analytes to be determined with the support under conditions with which binding is possible between the analytes to be determined and receptors specific therefore on the support, and
(iii) identifying those predetermined zones on the support onto which binding has taken place in step (ii),
(b) carrying out a subsequent determination cycle comprising:
(i) providing another closed support with a surface on an interior structure which comprises immobilized receptors on a plurality of predetermined zones, where the receptors in individual predetermined zones each have a different analyte specificity, where the receptors selected for the other support have been observed in a preceding cycle to be associated with a predetermined characteristic signal, and where the selected receptors or/and the conditions of the receptor-analyte binding are changed by comparison with a preceding determination cycle,
(ii) repeating step (a) (ii) with the other support and
(iii) repeating step (a) (iii) with the other support and
(c) where appropriate carrying out one or more further subsequent determination cycles in each case selecting and changing the receptors as in step (b) (i) until sufficient information is available about the analytes to be determined or/and until the signal meets a predetermined criterion after the binding event has occurred, the carrying of determination cycles including the integrated production and evaluation of a support in one instrument.

2. A method as claimed in claim 1, wherein nucleic acid analytes are determined.

3. A method as claimed in claim 2, wherein the nucleic acid analytes are selected from double-stranded DNA, single-stranded DNA and RNA.

4. A method as claimed in claim 2, wherein the nucleic acid analytes are fragmented sequence-specifically or/and non-sequence-specifically before the contacting with the support.

5. A method as claimed in claim 4, wherein nucleic acid fragments with a predetermined distribution of lengths are generated by the fragmentation and, where appropriate, a subsequent fractionation by length.

6. A method as claimed in claim 4, wherein nucleic acid fragments with an essentially homogeneous distribution of lengths are generated.

7. A method as claimed in claim 1, wherein the analytes carry labeling groups.

8. A method as claimed in claim 7, wherein the labeling groups can be detected optically.

9. A method as claimed in claim 7, wherein fluorescent labels or/and metal particle labels are used.

10. A method as claimed in claim 1, wherein the receptors are selected from polymeric probes.

11. A method as claimed in claim 10, wherein the change comprises an extension of the probe sequence.

12. A method as claimed in claim 10, wherein the change comprises an extension of the probe sequence.

13. A method as claimed in claim 10, wherein the change comprises a variation in the probe sequence.

14. A method as claimed in claim 1, wherein the change comprises a variation of the position or/and density of receptors on the support surface.

15. A method as claimed in claim 1, wherein the change comprises a variation in the nature of the coupling of receptors on the support surface.

16. A method as claimed in claim 15, wherein linker molecules used to couple the receptors are varied.

17. A method as claimed in claim 1, wherein the change comprises a variation of the conditions for binding between analyte and receptor.

18. A method as claimed in claim 17, wherein the hybridization conditions are varied in the case of nucleic acid analytes.

19. A method as claimed in claim 1, wherein the change comprise a variation of the synthesis conditions for constructing the receptor on the support surface.

20. A method as claimed in claim 1, wherein the change comprising a variation of the site geometry, in particular the size of the sites.

21. A method as claimed in claim 1, wherein the change comprises an empirical selection or specific selection of a receptor library.

22. The use of the method as claimed in claim 1 for differential expression analysis.

23. The use of the method as claimed in claim 1 for differential genome analysis.

24. The use as claimed in claim 23 for identifying chromosomal polymorphisms or aberrations.

25. The use of the method as claimed in claim 1 for the selection or/and optimization of hybridization probes.

26. The use of the method as claimed in claim 1 for diagnosis, e.g. for individualized or/and multistage diagnosis.

27. The use of the method as claimed in claim 1 in expression analysis for the selection of a subpopulation of genes.

28. The use of the method as claimed in claim 1 for the selection or/and optimization of capture probes.

29. The use of the method as claimed in claim 1 for the selection or/and optimization of antisense oligonucleotides.

30. The use as claimed in claim 1 for the selection or/and optimization of functional nucleic acids such as ribozymes.

31. The use as claimed in claim 1 for assisting or/and speeding up the selection methods in selection processes such a phage display.
